(19) United States
(12) Patent Application Publication
(10) Pub. No.: US 2023/0248856 A1
(43) Pub. Date: Aug. 10, 2023

(54) INDUSTRIAL HIGH-THROUGHPUT BIO-SCANNING

(71) Applicant: Richard Postrel, Miami Beach, FL (US)

(72) Inventor: Richard Postrel, Miami Beach, FL (US)

(21) Appl. No.: 17/609,151

(22) PCT Filed: Jun. 8, 2021
(86) PCT No.: PCT/US21/36277

§ 371 Date: Nov. 5, 2021

Related U.S. Application Data
(60) Provisional application No. 63/035,698, filed on Jun. 6, 2020.

(51) Int. Cl. A61L 2/07; A61L 2/22

(52) U.S. Cl. CPC A61L 2/07; A61L 2/22; A61L 2202/122; A61L 2202/14

(57) ABSTRACT

The present invention provides a platform, system and devices for testing or screening individuals for symptoms of disease, exposure to chemicals or biohazards. The device may be configured similar to the appearance of familiar airport screening devices, but may be configured for use in a people moving device or a walkway. The present device assays volatile compounds, especially volatile organic compounds (VOCs) emitting from a subject person, group or item. Different VOCs are produced dependent on metabolic changes in response to pathogenic, chemical, or biological stresses. Nanosensor elements within the device provide assessments of multiple VOCs to the platform with its integrated analysis capacities that rapidly compare instant data to a developed database. Each chamber may participate in an interconnected system sharing data for continuous improvement of assay procedures and comparison results.

The present invention provides for a walk-in or walk-through chamber or zone that rapidly and non-invasively screens individuals for disease, exposure to contaminants, chemical agents, or bio-threats, etc. The high-throughput bio-scanning process and devices are adaptable for a broad range of operations including, but not limited to those associated with: mass transit, airports, ferries, train stations, bus terminals, buses, vans, cruise ships, theaters, hospitals, military installations, factories, maritime vessels, embassies, sporting events, festivals, symposia, polling stations, food processing facilities, supermarkets, workplace environments, court houses, mobile clinics, space stations, deep space ventures and shuttle craft, etc. The predominant improvement comprises a series of novel applications of nanosensor technology necessary for the commercialization of devices to assay volatile compounds with appropriate, sensitivity, speed and accuracy. The present invention enables rapid detection of volatile compounds, especially volatile organic compounds in a time and cost-effective manner necessary for commercialization of high-throughput operations. The device has applications in a variety of fields including, but not limited to: immigration screening and customs, border patrol, coast guard, homeland security alcohol tobacco and firearms, postal inspection, capital police, World Health Organization, National Institutes of Health, Centers for Disease Control, Food and Drug Administration, health assessment, toxic exposure, biohazards, non-metallic explosives, past locations and exposures of clothing or persons or other items being assayed, drug impairment screen, etc. The methods and devices of the present invention allow rapid nano-assay of compounds emitted even in extreme low concentrations from a person or item of interest (hereinafter simplified to “subject”) within the scanning area or chamber. An item of interest may comprise a personal item, a container, package, etc., of assay interest. For example, items may be screened at ports of entry, airports, etc., especially at locations where a desire to screen persons is manifest. The nanosensor elements are capable of extremely dense packing to allow assays of an exceptionally large plurality of volatile organic and other compounds.

### BACKGROUND

Security concerns have led to development of touch-free frisk devices, such as metal detection and radio signal detection wands, as well as full body scanning devices. Since the early 1970s many airports and other locations wishing to screen for weapons, e.g., courts and legislatures installed initial screening protocols involving walking through a short metal detection tunnel. Persons who were flagged by this initial screen then were screened with a wand device to locate the source of the alert. A preferred configuration is one similar to those used in airport security where ionizing radiation is applied to produce an image. Though the shape in certain embodiments may be similar, the present invention differs in that it does not require exposing the test subject to even a minimal exposure of ionizing radiation.

The metal detectors in conventional devices rely on an electric pulse inducing an electric field. When a metal object is present in the field recovery to ground state is slightly delayed. Commercial devices may cycle through 25 to about 1000 Hz. In general, an alert requires a sustained series of relaxation anomalies. A relatively weak alternating electric-magnetic field. i.e., non-ionizing radiation is used in these devices.

With the rise of non-metallic weapons, including possible bombs, more sophisticated scanners were inserted into airport and other high security screening programs.

A first type consists of millimeter wave scanning whereby relatively long (low-energy) invisible light is used to image surface features on a body. The light in such machines may be in a range of 1 to 10 millimeters—about 1000 times the wavelength of visible light. The long wavelength allows the light to pass through most fibers such as clothes but is reflected off skin and other relatively dense objects such as buttons, belts, bottles, liquids and contraband. The image is produced by a rotating transmitter/receiver rotating and scanning the body within the chamber. Software, either man-made or improved by application of machine learning forms an image of the scanned object including surface anomalies such as concealed packages or suspiciously shaped metallic or non-metallic objects.

A second type involves backscatter x-rays. These are low intensity devices that monitor x-rays reflected back from the object they impact. These devices cause a minuscule increase in ionizing radiation exposure which has been expressed as a concern by those protesting the device. The exposure from a typical airport is equal to about 1/5,000 of a chest x-ray or a transcontinental or transatlantic flight. The backscatter image can reveal items with high liquid content, especially useful for security concerns, and can reveal shapes of items hidden by clothes (including body parts). While the exposure non-natural levels of x-rays is a concern to some, the specificity of imaging, including artificial joints, breast and facial implants, and details of all body parts has raised privacy concerns.

A third type rarely used is conventional x-ray where penetrating beams that pass through the bod are recorded at exit. These penetrating machines use x-rays similar to those used for medical imaging. But as we know from the lead shields often employed in doctors' offices, these rays are considered damaging and therefore to be used with caution when the benefits exceed the potential damages.

The present invention includes adaptations applicable to people in transit, for example a revolving door that may feature a diameter expanded beyond the common four divisions to lengthen time in a subdivision and possible provide a plurality of egress ports dependent on subject destination. While maintaining an appearance familiar to the subject walking through, the device can screen or assay mobile crowds for compounds of interest. A further adaptation of a familiar people mover involves embodiments where the chamber is essentially an open chamber such as a moving walkway or standway common in airports and train stations but with sensing capabilities surrounding the subject.

The background of these devices is provided for context. The devices of the present invention, while, in certain embodiments, similar in shape and appearance, operate using totally different monitoring principles. Concerns relating to these conventional devices therefore are not generally applicable to the presently disclosed scanning procedures and device capabilities.

### BRIEF DESCRIPTION

The present device scans a person, group(s) of persons, or item(s) of interest (collectively referred to as “subject”) that is/are present in or passing through a zone or chamber. The scan highlights potential issues including, but not limited to those relating: to safety, security, individual health status, public health, etc. The scan results in one or more such relevant report(s) obtained by measuring and analyzing volatile organic compounds (VOCs) that have been emitted or offgassed from the subject. Reports may be targeted for distribution to the subject, a device operator or monitor, local authority, and/or a remote authority, to apprise them of any results of concern.

The chamber comprises sensor elements that detect and or quantify (assay) VOCs in the gaseous environment immediately surrounding, i.e., coming from or affecting the subject. The chamber may physically isolate subjects using tactile barricades or in some embodiments the barricade(s) may comprise a volume of ambient gas (generally air), including, but not limited to: moving gasses that flush across the subject, gases drawn in or vacuumed from the immediate surrounds, gasses streamed towards or puffed at or near, the subject, etc. The chamber device will assay gas emissions, generally volatile organic compounds (VOCs), produced by individual subjects or groups of subjects within the defined chamber zone. The chamber design features nanosensor elements (NSEs) aligned within the chamber so that the subject's infinitesimal, almost trace, volatile emissions are brought in contact with the sensors. The “nano” in nano sensor can have two connotations. On the one hand, the sensor may be extremely sensitive, sensing in the parts per billion (nano or 10⁻⁹) range or less. Generally, such sensors are constructed using nano-technology. Non-NSE sensors may also be present. Though such sensors may be extremely small, generally sub-micron (<10⁻⁶m) in size, nano-sensors may be designed to present a large sensing surface, e.g., to increase sensitivity, reliability, probability of contact with the assay target, etc. The actual effective sensing size can be nanoscale or can be expanded to cover multi-micron or larger thickness and area dimensions. For example, a minimum dimension may be in the nano range dimensions larger than 1 Ångström (Å) (0.1 nm).

Air or gas flows are controlled at the periphery or within the chamber to flush volatile compounds off the subject and into contact with at least a subset of the internal array of the NSEs. Directed flows may be modulated by any appropriate means including, but not limited to any one or a combination of: pressure, electrostatic charge, nozzle orifice and shape, etc. to provide the desired flow rates, zonal velocities, volume, shape (e.g., vortex). Such flow or flows may be directed to flush the chamber to remove background compounds from the assay volumes to reduce or minimize non-subject produced NSEs. Directing gas including the volatile compounds under assay to the NSEs may involve on or more of a plurality of stimulations. For example, an acoustic standing wave might be set to levitate and move the particles, e.g. to propel compounds away from the subject and/or to draw them into contact with an NSE; a photon momentum based or a plasma actuator based system may be used in some devices or device components; an electro-static system may be used to drive compounds or particles away from the subject and/or towards contact with NSE. A systematic raster scan will be applicable in many embodiments while some embodiments may benefit from spiral scanning. The design choice made by one skilled in the art will select from the variety of scanning mechanisms and techniques useful in the art to achieve a 3D representation of scan results. When a scan has a time component, for example, several breaths of the subject, the subject movement and/or detection data may be included to provide a 4-dimension visual, tabular, or other output.

In some embodiments, the subject will remain static, in a three-dimensionally enclosed environment similar to those seen in airport security. In some embodiments, the enclosure may be incomplete, e.g., having an open top (roof) or side panels. Several embodiments feature a mobile chamber or a chamber assaying a mobile subject. Chambers may be for groups, e.g., a people mover compartment for an individual or a group of individuals. A static chamber may be configured as a walkway or carriageway where a subject walks or is transported through one or more chamber zones. For example, at an entrance to a public area a person might step through a revolving door or a carousel or onto a moving standway or a moving walkway. As the subject person progresses, the subject passes through one or more chamber(s)/zone(s). Any zone where persons may pass through individually or in small groups, including, but not limited to: elevators, escalators, halls, tunnels or covered walks between buildings or sections, transit devices (open or closed cars or carts), may be outfitted with a sensing system.

When entering the chamber, the subject might pass through dynamic air brushing cleansing flows or air curtain barricades that force gas over and past the subject to refresh the air carried into the chamber. Another portion of the chamber comprises propelling and/or hoovering orifices that direct volatile compounds from the subject to sensor elements. Said systems for propelling or hoovering gases will generally direct from higher concentration or higher kinetic energy (higher pressure) locations to lower pressure zones. Elevated pressures may be in compressed air or gas canisters, chemically produced, heating, air compressors, and the like. The reduced pressures may be achieved using a vacuum device, chemical extraction, cooling, etc. To contact sensor with gas, gases may be moved by any effective means known in the art, for example, by a pressure differential, acoustic drive, electrostatic effect, plasma actuation, photon momentum, etc. On a standway, the chamber may progress along the path with the subject or may move the subject through one or more chamber(s). The chamber may encompass the entire height of the subject or may be limited to a selected subject zone such as below the knee, the torso, below the waist, above the waist, head, head and neck, etc. or may be limited to selected height zones, e.g., arbitrarily, one or more zones of 0.1 m and/or 0.25 m, e.g., near the base or floor: up to ˜0.1 m, ˜0.1-0.2 m, ˜0.2-0.3 m, ˜0.3-0.4 m, ˜0.4-0.5 m, ˜0.5-0.6 m, ˜0.6-0.7 m, ˜0.7-0.8 m, ˜0.8-0.9 m, ˜0.9-1 m, ˜1-1.1 m, 1.1-1.2 m, ˜1.2-1.3 m, ˜1.3-1.4 m, ˜1.40-1.5 m, ˜1.50-1.6 m, ˜1.6-1.7 m, ˜1.7-1.8 m, ˜1.8-1.9 m, ˜1.90-2 m, ˜2-2.1 m, ˜2.1-2.2 m, ˜2.2-2.3 m, ˜2.3-2.4 m, ˜2.4-2.5 m, ˜2.5-2.6 m, ˜2.6-2.7 m, ˜2.7-2.8 m, ˜2.8-2.9 m, etc., ˜0.25-0.5 m, ˜0.5-0.75 m, ˜0.75-1 m, ˜1-1.25 m, ˜1.25-1.5 m, ˜1.5-1.75 m, ˜1.75-2 m, ˜2-2.25 m, ˜2.25-2.5 m, ˜2.5-2.75 m, ˜2.75-3 m, etc., from the base or floor. The one or more zones may include one or more zones ˜0.1 m in length and one or more zones 0.25 m in length or may be designed to differentiate to any desired increment(s). The full height is designed for the intended use, e.g., for humans a fraction over 2 m would be adequate. The “height” zone discussion would be applicable to horizontal zones when the chamber is configured in a horizontal format an accordingly may we exceed two or three meters, for example in a walkway or standway. A “height” perpendicular to the length of assay area may feature limited zones similar to the example zones listed above.

Such chamber may have one or more open sides or an open top or may appear in a tunnel format.

The chamber hardware will incorporate or have access to at least one analytical/computational system that can store the assay information and, when present, data from non-NSE sensors or other source inputs which may include, but be not limited to: manually or automatically inputted data, results of assays other assays, conditions of the assay, such as mass of subject, steps/stages of assay processes, chamber integrity, chamber functions activated, chamber and chamber components movements, volumes in flux, fluxes where interacting with sensors, specific NSEs activated at any stage, operator identification, subject movements, subject identifying information, subject health information, subject temperature, time of encounter, elapsed exposure time, etc.

A preferred format incorporates a ring or rings inside a chamber to transport NSEs. Gases may be vacuumed, preferably through a port positioned a few centimeters off a portion of the subject surface. Gas may be replaced locally, e.g., by streams or puffs of gas released proximal to the vacuum port and delivery gas released from the puffed location, or more universally, e,g, through ports in the floor, cap or walls. The scanners may incorporate one or more lights to indicate where the gases are being harvested. Lights may also be used alone or in conjunction with fluoroactuators to scan for specific hazards, such as bacteria, targeted chemicals, nucleic acid, blood, etc. A ring may be open, e.g., shaped as an arch or doorframe, or closed, e.g., circular ovoid, elliptical, etc. Aesthetically and for simplicity in shape, the chamber will be a cylinder slightly larger in diameter than the ring. However, the shape of the chamber is not an essential feature of many embodiments of the present invention. A box shape, ellipse, hexagonal, pentagonal, octagonal, triangular, oval, circular, a geometric shape of Non-Uniform Rational Bicubic Splines (NURBS), or other shapes may be used. The term “oval” refers generally to a closed curved shape that may be point symmetrical like a circle or linear symmetrical like an ellipse or egg. The term may also include similar shapes that may be interrupted in portions by one or a plurality of gaps. The gap may appear as an opening or may connect to a portion projecting out of the main plane of the oval. The ring itself also is not limited to an oval or circular shape but can be of any suitable shape accommodatable in the chamber. The ring as understood herein refers generally to a border for presenting the NSEs to a subject area. It may be a curved structure, but may feature linear segments. It may be fixed size or may be adjustable to accommodate the size of the subject. The ring may be fixed or may be rotatable within the chamber, may follow a vertical path, may be regular or irregular in shape, may be parallel or at an angle to the base and may comprise a single unit or include coordinating components. A ring may be a series of parallel or semi-parallel strips that ring the subject. The ring or probe may make a single pass, e.g., from top to bottom, bottom to top, once around clockwise or counterclockwise. The sensors make make multiple passes, e.g., reversing and/or repeating a scanning circuit. Software may interactively control the rate of movement at any portion and/or the need for reverse or repeat scans of the whole subject or identified portions. A user interface may be integral or remote.

NSEs carried on the chips can be any properly designed sensing surface capable of, for example, field-effect transistor (FET) or other physico-electrical property/activity including, but not limited to: semi-conducting nano-wires, carbon nano-tubes—including single-wall carbon nano-tubes, chitosan-cantilever based, synthetic polymers—including dendrimers, plasmon resonance nano-sensors, Förster resonance energy transfer nano-sensors, vibrational phonon nano-sensors, optical emitting, optical frequency (or wavelength) based nano-sensors (sensitive to photon transmittance, absorption, reflection, energy modulation, etc.). NSE devices can be constructed from different formats, including, but not limited to: conductive polymers, single wall nanotubules, graphene, etc. Nano FETs and other nano-sensor formats generally operate by changing electrical properties as a substance comes in close proximity to the sensor by perturbing the steady state (absent the proximal substance) charges and movements (distribution of electrons) within the nano-sensor. A transistor's effective electrical properties causing an observable change in electron flow (current) sensor competence is manifest. A transistor is one example of NSE format. The present invention is not restricted to this example, but may advantageously use other electronic control devices including, but not limited to: a 2 terminal, a 3 terminal, a 4 terminal, etc. electronic signal device's sensing signal. The altered distribution of electrons, depending on the design of the nano-sensor, changes one or more electrical properties, e.g., impedance, resistance-conductivity, capacitance, inductance, etc. and thus the physical movement of a detectable particle, e.g., an electron, a photon, a phonon, a bipolaron, etc.

The chamber provides an enclosed or an isolated environment that isolates the subject for assay. Enclosed embodiments feature a chamber that has at least one opening that allows a subject to enter and to leave when desired. When closed the opening isolates the subject from the external environment. The opening form may be any available design function, including, but not limited to: lifting the entire chamber sides to allow a subject access to a base inside the chamber, lifting a portion of the chamber that acts as a door, sliding vertically or horizontally a portion of the wall of the chamber to act as a sliding door, opening the chamber with horizontally aligned hinges to raise or lower a door, opening the chamber horizontally with vertically aligned hinges, etc.

Open chamber embodiments such as a moving walkway may be open or covered at the top. A cover need not be continuous. For example, an arch or arches along a walkway may constitute a sensing station. Multiple stations along a walkway may make similar assessments differentiated by time and/or may concentrate on a different grouping of VOCs. A person may stand on the belt or may walk. Belt systems may feature labels such as those used to guide social distancing and to disperse or separate persons to allow individualized assessments. When used as a walkway strip, signal lights may be used to suggest pacing. For example, when a person might be overtaking another or simply approaching too close, an audible alarm may sound, and the streaming lights may change color to red or flashing red. In more rigorously controlled environments, applications that employ signaling familiar subjects may instruct subjects how to proceed. For example, where persons are familiar to traffic signaling: go, caution, and stop colors, e.g., green, amber, and red lights may be used.

In some embodiments, such as a moving platform or transport carriage, overhead straps may be featured to separate individuals and may be put to additional advantage by inducing movement of the arm and releasing VOCs. This practice may also be of interest when VOCs from an area such as the torso or underarm are relevant. While being transported along a pathway, visual or audible instructions or other media may be accessible, e.g., cartoons, speeches, news-feeds, videos, advertising, concerts, etc.

The chamber is self-contained in that it minimal affects or does not affect ambiance of the external environment during assay procedures. Since the chamber is self-contained, pressure within the chamber is independent of the ambient temperature; inside may be the same as, positive, or negative with respect to the outside. Humidity may be modulated by controlled flow of chamber gasses, temperature control, humidification and/or de-humidification. Some versions of the chamber may include adaptable lighting, perhaps to calm or to comfort a subject or to emphasize or highlight one or more features, e.g., timing of the assay, physical area being targeted, portion of the ring being activated, providing instruction to operator or subject, indicating progress or function, fluorescent tags, etc. Sound dampening using active or passive noise reduction may also be featured. The chamber may feature a sound and or video system that may be operator or machine controlled, for example to advise or instruct the subject. For example, if data being to sense subject discomfort, an operator might be alerted to speak to and hopefully reassure or calm the subject. Such response may be automated with a set of queries or reassurances or may merely be instructive to a human operator. For better exposure to certain body areas, perhaps to more precisely identify a source of a VOC, the sound system may ask the subject to perform certain movements or tasks, such as bending knees, lifting arms, leaning forward or back, holding breath, breathing rapidly, etc. A system may provide an oral version of a medical history questionnaire where while the scans are progressing, the subject is distracted by responding to questions. Such questions may be helpful in establishing baselines when the chamber is testing reliability (truthfulness) of the subject's responses.

A system to map a subject in 3 spatial dimensions may be incorporated into a report so that a graphical presentation showing source or various detected compounds in available. While a detailed image is potentially available, for example, by using magnetic resonance or CT scans, monitoring the locations through which the VOCs escape the skin or body orifices may obviate the need or desire for inconvenient and expansive internal topography. A digital camera moving vertically and horizontally around the body provides sufficient detail in multiple 2D images for a stereoscopic reconstruction of the entire surface when photos from multiple angles are present. An alternate method uses air flow changes in contact with the body to provide a three-dimensional rendering of the body's surface. When combined with the site specified source(s) of volatile compounds the subject, operator, or other interested person(s) can visually interpret sources of each identified compound or class of compounds. The part of the body emitting the VOCs, e.g., hand, jacket, foot, head, etc., may suggest a potentially hazardous item of clothing should be discarded, indicate portions of the subject that need additional screening or cleansing, suggest where the subject may have been exposed, etc. Some embodiments may feature sensors at the base to concentrate on the feet. In some circumstances it is understood a subject may be asked to remove shoes. Sensors may be positions at the base and/or gases hoovered through the base may be directed to more centrally located sensors.

Human subjects may exhibit anxiety or other discomfort in an enclosed environment. Adaptable lighting as referenced above is one potential tool for reducing anxiety. The NSEs in the chamber can be used to provide feedback as anxiety associated VOCs are monitored. The device may cycle through feedback processes to reduce such discomfort or anxiety. In some embodiments VOCs such as spice smells may be introduced. These scents may be used in feedback processes to optimize subject comfort. Spices including, but not limited to: peppermint, cinnamon, chamomile, lavender, lemon, sandalwood, rosemary, jasmine, bergamot, vanilla, Christmas tree, etc., have been suggested for calming applications. The device might also be applied in a scientific setting to monitor VOCs in response to an event under study, including, but not limited to: a frightening movie scene; a sound such as: a tone or tune, a selected pitch or chord, a language, etc.; food fragrance; color schemes; pollen; insect parts; animals; dander; suspect foods; etc.

The device may sense subject discomfort relating to social interactions, such as fear, lying, satisfaction, etc. In cases where, for example, fear or anxiety is sensed an adapted assay procedure may be followed. Such adaptation may be operator determined or suggested or may be in accordance with a predetermined automated algorithm walls may turn opaque. Anxiety levels may guide questioning an individual, e.g., intelligence interrogations, customs. Machine learning algorithms may be applied to sense metabolic events associated with a person's uncertainty or prevarication in response to operator or machine instigated queries. The algorithms are potentially updated periodically by applying data obtained from the device. For example, sensitivity can be adjusted for each NSE or for any subset of NSEs in accordance with self-calibration principles. The experience of a single device may be used to calibrate other functions, such as ideal rate of movement, pulsing, etc., of a movable NSE or intensity of gas propulsion or strength of vacuum. Updates may be periodically distributed as is common with modern electronics. Individual devices may be interconnected in a data-sharing system preferably available for artificial intelligence/machine learning technology that may continuously send messages or alerts to relevant recipients, perhaps recognizing inefficient or faulty coding, bad batch of product used in the machine, maintenance issues, etc., but possibly more usefully to signal patterns of potential new disease threats at a very early stage, even when characteristics causing or underlying the anomaly are anecdotal.

In a diagnostic mode, the chamber may be used to rapidly assess responses to environmental influences, e.g., introducing a compound, smell, stimulus (light, sound, odor, etc.) to screen for contributory factors or causes of allergies, migraines, seizures, anxiety, sleep, etc. The chamber when used in this manner can provide early diagnostic screening methods that would greatly improve a patient's experience by eliminating many invasive and prolonged testing regimes. The result will be greatly improved patient comfort and outcomes.

Such device may also have applications for screening visitors to a home or business for a rapid assessment of potential contagion, avoidance of allergic response, identification of repeat visitors, potential association with hazardous materials, etc. Some embodiments may be configured to accept persons assisting the subject.

The chamber may allow the subject to be visible or may be translucent or opaque. The opacity of the chamber may be switchable.

A gas is flushed or otherwise caused to flow through the chamber to purge, to remove or to clear compounds that enter the chamber with the ambient atmosphere.

Flushing/clearance may be accomplished in a manner chosen by the operator or device designer. For example, the base may include ports that exhaust cleansing air/gas with the exhaust hoovered up by a relative vacuum (reduced pressure) at the top of the chamber. The top and base are arbitrary as the flow could be in an opposite direction. Flushing may be accomplished by a motile or dynamic cleansing operation. For example, a ring or rings (or other shape frame) may be traversed from top to bottom or bottom to top; a vertical oriented rod may rotate around the chamber; a rod may take on a central position delivering a flush gash to be hoovered at the periphery, etc. A ring may collect or exhaust the clearance gas. A ring may be static or may traverse the chamber. A pair or plurality of “rings” may cooperate to exhaust and hoover the gases. A ring may be multi-functional, e.g., exhaust at its bottom or top region with the hoovering and sensing displaced above or below. An edge of the sensor band, e.g., a ring may serve an air curtain to exclude or minimize systemic, random, or pseudo-random variability. The ring may include multiple bands of sensors. The ring system may be constructed as a single piece or may include separate bands above or below other rings.

The chamber may feature an armrest, strap or handle so that a person can stabilize their stance while standing. When the armrest, strap or handle is positioned so that the person raises at least one arm to expose the side torso and underarm or armpit zones, VOCs from these zones may be advantageously assessed. The armrest strap or handle can be designed to provide heart rate and pulse monitoring and may be outfitted to read blood oxygen.

A sterilization feature between subject assays may be accomplished using any process that does not damage or hinder chamber function. For example, a sterilizing gas may be directed against surfaces or may be spread throughout the chamber to avoid cross-contamination between subjects. A misting may be used to distribute or deliver active ingredient. One conventional example involves ethylene oxide. Heat is also available in some embodiments using heated gas and/or heating elements in chamber components. Steam is an especially effective heat sterilization technique. Ultraviolet light, especially UV-C, short wavelength, ˜200 nm to 280 nm light is less damaging to human tissue than the longer wavelength ultraviolet subdivisions, UV-A and UV-B. Aldehydes are also frequently used as disinfectants, especially for periods in excess of 10 minutes. These must be used with caution to avoid risk to humans and are not a preferred disinfectant. A reduced pressure (vacuum) may contribute to removal of cross contaminating volatiles and/or to serve as a quality check on chamber integrity.

Ethylene oxide (EtO) gases are advantageous where heat, humidity or coating or bathing with liquid, is difficult or may be avoided due to potential damage to components being disinfected or sterilized or potential adverse impact to people or items in proximity to the chamber. EtO diluted with CO₂ is an effective sterilant, while the CO₂ with humidity or steam also contributes to acidic sterilization. H₂O₂ and ozone are gaseous disinfecting oxidants available for use as mists or vapors.

Disinfection using heat has an additional benefit of increasing vaporization or the off-gassing of volatile compounds. Heating between subject assays thus is an advantageous factor for reducing carryover from one subject to the next. Temperatures above body temperature (37° C.) are preferred. Increased chamber surface or internal temperatures, e.g., the chamber, components or portions of the chamber a maximum or threshold may be brought to a desired temperature such as examples found in literature, including, but not exclusively limited to: about 40° C., 42° C., 45° C., 50° C., 55° C., 60° C., 63° C., 65° C., 67° C., 70° C., 75° C., 80° C., 85° C., 90° C., 95° C., 97° C., 98° C., 99° C., 100° C., 102° C., 104° C., 105° C., 120° C., 150° C., 180° C., 200° C., 230° C., 250° C., 270° C., etc. Different components of the chamber may be heated to different temperatures. Steam may provide the heating or may be used in conjunction with heating, for example, resistance heating. Heating may be convective from controlled streams of gas, mist or steam; may be inductive, may be resistive or may be accomplished using light, often an infrared (IR) source whose wavelength distribution and peak wavelength are chosen for correspondence with the absorptive, reflective and transmission characteristics of the coating or substrate surface being heated. Drying may be effectively accomplished using IR in about the 3000 to 5500 nm range and about the 6500 to 10,000 nm range. Water tends to absorb in this range and thus evaporation will not be dependent on the wet surface substrate absorptive properties. Emission in a range of from about 5500 to 10,000 nm is effective for sanitizing or disinfecting a surface. Shorter wave IR may be used, for example, from about 1700 to 4000 nm when proteins or lipids are preferred targets. As a guideline the —OH⁻¹ (hydroxyl) group especially present in water, sugars and short alcohols absorbs from about 2700 to 3300 nm; the C—H bond, present in lipids, sugars and proteins absorbs from about 3250 to 3700 nm; and the N—H bond (proteins) absorbs from about 2800 to 3300 nm. An IR and UV source may be used together.

UV-C is preferred for decontaminating/disinfecting because although this C-band is strongly absorbed and damaging to nucleic acids and thus lethal to propagation of microbes exposed to the light, this band is strongly absorbed by skin and fails to penetrate beyond the outer (dead skin) layer of humans. A mercury-based lighting source is available with a spectral emission about 254 nm, well situated in the UV-C band.

In several circumstances, alcohols including, but not limited to: methanol, ethanol, isopropanol, etc.; acetone; and/or other sterilizing or cleansing liquids may be used alone or in combination when desired—with proper safety precautions following approved safety and exposure protocols.

A flushing feature may include a liquid or mist. The liquid may be used to sanitize or decontaminate the subject. A mist may deliver a dissolved gas as an active ingredient. A controlled gas flow may be used to remove or to dry the applied liquid or mist. Gasses may be air, a controlled gas mixture, a purified gas, etc. If the subjects head is isolated for breathing or if a mask is provided, the gas may be microbicidal, inert and oxygen free.

Following the flushing process, the assay will include fewer results from volatile compounds in the surrounding atmosphere. A second flush or a plurality of subsequent flushes may be initiated to reduce background more thoroughly, such as may be carried in subject's clothes, hair, etc. In some embodiments the subject will be analyzed “as is”, e.g., in street clothes; the subject may be gowned; the subject may be slightly dressed or undressed as if for a shower.

Exhaust gas may be pulsed, continuous, cycling, or otherwise perturbed (e.g., stochastic or pseudo-stochastic). If continuous, the flow rate may be constant. The flow may be unidirectional or may be configured in a vortexing pattern. The software (algorithms) can be devised to deliver location information.

A mobile frame may transport NSEs selected to assay one or more VOC(s) or other volatiles of interest. A mobile frame may provide an exhaust directed at the subject to drive volatiles from the subject, for hoovering in a frame. The hoovering and exhausting frame may be the same or may be physically distanced. The exhaust and/or hoovering may alternatively be located in a base or top or cap of the device. For example, the NSEs may be in a ring that draws in the ambient gas to contact the NSE surfaces, while the gas is replenished through the cap or floor.

The device may be multi-compartmentalized. For example, for a human subject, the head may be excluded form the analysis compartment either in a second compartment or left in the ambient atmosphere. When the head or mouth/nose is isolated in a separate compartment—possibly as simple as a mask—breath is available for independent analysis. The breath intake may also be controlled using a mask if controlling the inhaled content of breath is a desired feature. The mask may be used to monitor and or control inhalation and exhalation in a combined unitary format.

The device of the present invention provides rapid highly sensitive detection of VOCs in a gas phase sample. Analytical data are then processed using the device's library of algorithms to detect a disease or to answer questions for which the sample was taken. Especially when a device participates in an interconnected system it is continually developing and improving its algorithms through shared machine learning and artificial intelligence. The system may use non-linear system identification techniques (such as Volterra kernel representations) as a tool in this continual development.

In some screening situations, the assay may be targeted to identify a toxic substance, a potentially dangerous substance, or contraband. The assay results are compared with a database with sample results being alerted according to the screening purposes/criteria. For example: the screen may indicate a traveler visited a farm and might be subject to additional screening for agricultural disease; the screen my identify fuels or organics correlated with explosive manufacture; the screen may flag one or more diseases associated with the VOCs detected; the screen may be periodic and be used to assess disease progression or therapeutic efficacy; etc. The system may include access to domestic, international or private databases, for example, to correlate or include medical information for documentation or improved analysis; the system may receive personal or group demographic data used to catalog, analyze and possibly contribute current subject data back to the accessed database; in some screenings law enforcement data may be used to recognize correlative findings between assay results and previous illegal or legally monitored activities; travel, immigration, address and/or residence databases of the present subject or compiled data may improve categorization and analysis of VOC results. In some circumstances, the system may acknowledge and advise the sending database of non-medical assay results—e.g., for law enforcement, health tracing, etc. Job descriptions, group demographic, personal demographic, occupation (including diseases associated with occupations), previous residences (for example at high altitude), may expand useful analyses of present data. Financial, banking government benefit status and insurance information may be relevant in specific circumstances.

Through capturing the VOCs in vapor or gas phase to measure the presence, amounts, volume, intensity or strength of signal of multiple VOCs, then classifying each signal as from the organism or the environment and removing foreign VOCs from analysis consideration, the device then outputs a sample's gross output of organism initiated organic compounds for comparison to the signature database to determine whether a specific disease (or set of diseases) is present. The present invention continues to consolidate VOC signature profiles into a library as new sample outputs are presented.

An alternate application of the present device and assay processes is as a screen for exposure. For example, if possible, exposure to a toxic volatile is of interest, a simplified assay may be programmed to detect the toxin of interest or its metabolites. In some embodiments, metabolic responses to exposure my be assayed to determine, for example, exposure levels and how the subject is handling (metabolizing) said toxin and expected dates of clearance.

Embodiments may include accessory features or sensors such as subject identification. Facial, hand, gait, or other bio identifier may be incorporated into a device to accomplish multiple tasks with a single mechanical interaction. A simple count of subjects, possibly with a report or graph of volume of reads during the day, week, or month, etc., may guide device maintenance, staffing requirements, etc. For example, a sudden spike in count may call for an additional path to be brought online. Demographics, including, but not limited to size, height, mass, sex, time for scanning, speed of movement, a photograph, apparent group size, etc. These may be useful for purposes, e.g., lost child, missing elder, person of interest, expected requirements farther down the route, e.g., walkway or path being followed.

The scanning device may be put to additional functions not directly relating to a subject scan. When not actively scanning, the device may constantly or intermittently monitor ambient conditions. For example, if total VOC content or content of specifically problematic VOCs are outside limits, ventilation may be increased, a corridor may be closed, a safety crew dispatched.

In confined or controlled environments the device may be outfitted to monitor, O₂, CO, CO₂, N₂, nitrogen oxides, active oxygens, etc., e.g., meter turnover with outside air or stored gases to assure that air breathed by crew or helium sensor and meter may be used in high pressure, especially deep sea explorations. The device with one or several accessories can contribute to facility efficiency by meeting multiple needs or wants with a single scanning and monitoring unit.

In a longer range, less high-throughput application, clothing may be flushed and assayed, followed by a second flush/assay to detect emission from the skin, breath, etc. of the subject.

An alternate configuration may use vertically arrayed static strips. Input of moving gas may be from a diverse source such as from the base or cap. Input may be spatially controlled so as to direct continuous or pulsed flow over a progressing portion of the subject over time. The hoovering may coordinately progress in a manner offset from the inflows to collect data progressing spatially over the entire subject. The levels of feed and hoovering may be offset angularly such that the exhausts and hoovering are offset horizontally; the exhaust and hoovering may be offset vertically. Software aware of these timings and offsets will aid in locating the spatial source of the VOCs.

Vertical stripping may include NSE strips in parallel or in combination with lighting strips, directed flow orifices or strips, etc. The strips may be static (not moving with respect to the chamber when a subject is present) or may be rotated, for example in a counterclockwise or clockwise direction as selected by the chamber designer, software engineer, operator, or machine learning based algorithm around a subject. In some embodiments, the strips may remain static while a subject is manipulated within the chamber, e.g., in accordance with machine or operator instruction to the subject, rotation or partial rotation of the chamber base, etc. A subset of strips may be activated by machine or operator in concert with a predetermined schedule or in response to assay events.

Vertically arranged NSEs may be used in a supportive role in conjunction with one or more sets of horizontal NSEs. For example, the vertical NSEs may activate during a flushing operation following subject enclosure in the chamber. The vertical sensors might monitor the flushing and signal when a stasis condition, e.g., readings indicating successful flushing, occurs. The signal could signal the horizontal sensors to begin monitoring the subject. Mobile ring(s) may rise from the base or descend from the top, preferably directing air towards the subject while another portion hoovers subject related gaseous content past the NSEs. For quality control the vertical stripped NSEs might remain operational during this assay function and may, in accordance with a predetermined algorithm, modulate the rate of movement of one or more assay ring(s), select individual NSEs or subgroups of NSEs to be active, adjust sensitivity of an NSE or subgroup of NSEs, adjust amount of gas inputting or intensity of vacuuming to optimize chamber performance. The vertical movement may be reversed in response to collected data, perhaps to confirm or refine analysis by rereading a subject body area as a repeated assay with or without sensor adjustment.

The device may be used periodically by or on a specific subject to, for example, provide a timeline showing changes such as disease progression or efficacy of treatment activities. The period between such assessments is determined in accordance with desires/needs of a therapist and/or subject. Where relevant, a daily assessment may be made using a device in the home. This might be used in analogy to a person stepping on a scale every morning. The device may include additional measurement or acknowledgment features such as a scale, a radiation detector (Geiger counter), body fat, O₂ consumption, temperature, x-ray, heart rate, etc. Such daily or otherwise frequent check may be used for example, to plan the day's activities or to address nutrient deficiencies or excesses.

Some embodiments feature an interconnected system of devices, for example, devices sited in a city, county, state, etc.; immigration authorities, machines controlled by a specific group, e.g., a hospital; a hospital system; a company's sites; etc. Together such interconnections form a more robust machine learning platform that may identify similar anomalies across a plurality of machines. Data collected over the interconnected system would be merged and continuously analyzed to recognize new patterns. Disease detection would be accelerated as these anomalies are flagged. This rapid recognition of previously unseen, unrecognized or unknown medical anomalies can flag sites or individuals for follow-on screening. The large data sets can be analyzed with anonymity with subject location or identity only available in accordance with relevant regulations. This interconnected system is also applicable to obtain an early notice alarm in chemical defense or bio-defense platforms.

Devices of the present invention may be incorporated as part of triage operations. One configuration may allow the device to maneuver so that the opening communicates with a selected exit pathway or corridor. An alternative configuration may include multiple potential ports of exit from the cylinder. In such operation, such as following a large-scale exposure to a hazardous material or bio-hazard, the subject would enter the chamber. The chamber would seal. The internal portion would be flushed and the assay commenced. Depending on the assay indication, the machine software would provide an exit path for the subject to, for example, a containment or isolation center for those subjects flagged as exposed or unhealthy, a path for subjects not at risk or possibly just requiring a shower or change of clothing, or a path for those in a zone where further screening is suggested. The thresholds for such delineations can be set by a human or by algorithm developed in advance or in accordance with machine learning. A robust flush/sanitation/disinfection/10 decontamination may be practiced following assessment of a harmful exposure. A less robust flush/etc. may be practiced following assessment of lower risk. The selection of cleansing processes may be pre-determined, may be under operator control or may be interactive in accordance with a predetermined or adaptable machine learning based procedure.

Some embodiments feature a motile sensing station. The station may be collapsible or foldable for easy packing and transport. Portions may expand in accordance with any conventional packaging. E,g., the packing and unpacking involve manual manipulation; the packing and unpacking may be automated with little need for human input (a push button or remote instruction); the packing and unpacking may occur in a step wise process with human acquiescence (inspection); the packed or unpacked sensing device may include its own powered motility. One or more motors may serve to position sensing capacity in any of three dimensions. That is one or more motors may direct movement along two dimensions parallel to a liquid or solid surface and/or one or more motors may contribute to elevating sensing capacity above a liquid or solid surface. Motive power may assist the operator in manipulating and/or positioning the device. The device may operate independent of a clear line of sight of an operator.

The sensing component(s) may be self driving selecting targets for assay using artificial intelligence or may be under remote control. It may approach a select animate or inanimate subject in an active sensing mode or may activate during approach or upon arrival. It may be directed to a site to monitor approach or arrival of a subject. It may be limited in sensing scope, for example, sensing zones near the ground or around feet. It may project an arm to target a select area near or on the subject. It may be ground based, aquatic, or aerial in approach to a target subject. An aerial version may act as an aerial drone, that is, adopting a station above a subject or a path to be used by a subject. It may monitor gas above the subject(s). It may drop or project a probe to become more proximal to a subject. A drone may park on a solid support upon arriving at its destination. A drone may extend lateral or vertical support for stabilization at the targeted site. Sensed data may be analyzed onboard with data compared to a library downloaded into the drone device. Drone sensed data may be transmitted to a remote analytical device for completion of analysis and reporting.

Monitoring VOCs as accomplished using features of the nanosensing device opens the potential for interesting research. For example, individuals or focus groups may have their VOC production/emission responses monitored. A movie director or editor may wish to test which versions evoke more fear, disgust, warmth, satisfaction, excitement, or other emotion when editing, digitally modifying, or refilming a scene. An advertising agency may use the technology to assess marketing approaches. Trainers or educators could apply these principles to adjust training or teaching formats or timing. While not the original inspiration for the screening device these uses may prove revolutionary.

The device as described above is a preferred configuration as an upright subject (person) will present a maximal surface area for VOC emission to the chamber. For some purposes, the chamber may provide a surface upon which a subject may be laid flat and possibly rotated. The chamber itself may be maneuverable to accept vertical or prone subjects. The chamber may be configured with a capacity for wheeling a subject in or out. Another adaptation may feature a bedlike platform to position, for example, a baby or pet. Expansion of the general device to a wider subject area may be featured to accept groups or persons in wheelchairs or other supportive device. Capacity for a parent or companion to join the subject within the chamber is another design option. A foldable or removable feature such as a dropdown seat may be used for subjects discomforted by standing in the chamber or unable or to stand for the assay. A headrest or hand grip(s) is other option that may be incorporated to increase subject comfort. In some such embodiments, instead of top or bottom the reference may be more accurately characterized as “left” and “right”.

1. A chamber configured in size to contain a subject, said chamber comprising:
a barrier isolating an internal gaseous environment for surrounding said subject from the volume outside the chamber;
said barrier incorporating at least one portion to allow ingress and egress of said subject;
an internal array of nanosensor elements (NSEs) for sensing volatile compounds;
a gas directing mechanism that moves gas across NSEs in said internal array;
a system for collecting data from said NSEs; and
a system for compiling and analyzing said data.

2. The chamber of claim 1 wherein said volatile compounds comprise volatile organic compounds (VOCs).

3-9. (canceled)

10. The chamber of claim 1 wherein said gas directing mechanism comprises a system for propelling a gas towards said subject.

11-12. (canceled)

13. The chamber of claim 1 wherein said gas directing mechanism comprises a ringed shaped system for propelling a gas toward said subject.

14. The chamber of claim 13 comprising a plurality of ringed shaped systems, at least one of said plurality comprising at least a first array of NSEs, and at least one of said plurality comprising a system to propel a gas towards said subject.

15. The chamber of claim 1 wherein said gas directing mechanism comprises a system for feeding or drawing a gas across at least a portion of said array of NSEs.

16. (canceled)

17. The chamber of claim 1 wherein said gas directing mechanism that moves gas across NSEs comprises at least one motive force selected from the group consisting of: pressure differential, acoustic drive, electrostatic effect, plasma actuation and photon momentum.

18. The chamber of claim 1 comprising a field-effect transistor NSE.

19-21. (canceled)

22. The chamber of claim 1 wherein said system for collecting data comprises sensing movement of at least one entity selected from the group consisting of: an electron, a photon, a phonon, and a bipolaron.

23-25. (canceled)

26. The chamber of claim 1 wherein said portion comprises the chamber body that is elevated to allow said ingress.

27. The chamber of claim 1 wherein said portion comprises the chamber body that is elevated from below to barricade the subject.

28. The chamber of claim 1 wherein said portion to allow ingress and egress comprises at least one slidable portion of the chamber body that is lifted, dropped or rotated to allow said ingress.

29. The chamber of claim 1 in a geometric shape Non-Uniform Rational Bicubic Splines (NURBS).

30-32. (canceled)

33. The chamber of claim 1 further comprising at least one feature selected from the group consisting of: pressure control, humidity control, temperature control, noise reduction, controlled lighting, variable wall opacity, scent distribution, digital recording and/or playback, sound system, audio instructions, video instructions, body mapping (3D).

34. The chamber of claim 1 further comprising a system for sanitation, disinfection, or sterilization.

35. The chamber or claim 34 featuring a light source emitting in at least one wavelength in a range from about 200 nm to about 280 nm.

36-38. (canceled)

39. The chamber of claim 34 wherein said system for sanitation, disinfection, or sterilization comprises a flushing with a liquid or a mist.

40. The chamber of claim 34 wherein aid system for sanitation, disinfection, or sterilization comprises a flushing with an approved chemical and dosing protocol.

41. The chamber of claim 1 further comprising a mask apparatus to distinguish the subject's gas intake from the gas in the surrounding chamber.

42. The chamber of claim 1 further comprising a mask apparatus to distinguish the gas exhaled by the subject from the gas in the surrounding chamber.

43. (canceled)

44. The chamber of claim 43 wherein said mask apparatus to distinguish the gas inhaled by the subject from the gas in the surrounding chamber and said mask apparatus to distinguish the gas exhaled by the subject from the gas in the surrounding chamber are unitary.

45. The chamber of claim 1 wherein the barrier comprises a partition in a revolving structure.

46-47. (canceled)

48. The chamber of claim 1 wherein said barrier comprises a gas.

49. The chamber of claim 48 wherein said gas barrier is formed by spatial separation of subjects or an air curtain.

50. (canceled)

51. The chamber of claim 49 further comprising a moving standway or walkway.

52. (canceled)

53. The chamber of claim 1 wherein said internal array of nanosensor elements (NSEs) for sensing volatile organic compounds (VOCs) is zoned to access separate data elements from a plurality of zones.

54. The chamber of claim 53 wherein at least one zone comprises a torso or underarm area.

55. The chamber of claim 1 wherein said system for compiling and analyzing said data features a comparative system comparing a subject's data to a database, recognizing similarities, tagging and optionally ranking similarities and providing a report to an operator or designated report recipient.

56. The chamber of claim 55 wherein said system is dynamically updated following machine learning protocols.

57. The chamber of claim 1 wherein said at least one portion to allow ingress and egress comprises at least a first openable egress port and a second openable egress port.

58. The chamber of claim 53 wherein one of said first openable egress port and said second openable egress port serves as the ingress port.

59. The chamber of claim 57 wherein said system for compiling and analyzing said data is configured to select and open one egress port when predetermined criteria are satisfied.

60. The chamber of claim 59 further comprising a plurality of zones external to said chamber, a first of said external zones separated from a second of said external zones, access to said first and said second egress zones in communication with said first and said second egress port, respectively.

61. The chamber of claim 59 wherein said configuration to select features an interface with at least one NSE.

62. The chamber of claim 1 further comprising interface components that receive data from an external database.

63. The chamber of claim 62 wherein said external database is maintained by a monitoring agency.

64. The chamber of claim 63 wherein said monitoring agency is governmental or private.

65. (canceled)

66. The chamber of claim 64 wherein said database comprises data types selected from the group consisting of: medical, law enforcement, travel, immigration, residence, personal demographics, group demographics, occupation, international relations, banking and finance.

67. The chamber of claim 64 wherein said database comprises data types selected from the group consisting of: employee files, job descriptions, medical, travel, residence, personal demographics, group or team demographics, occupation, previous occupations and insurance.

68. The chamber of claim 1 further comprising interface components that receive information from a system that has compiled received data from a plurality of devices.

69. The chamber of claim 68 wherein said information is continuously updated.

70. The chamber of claim 1 further comprising interface components that transmit data to a system receiving data from a plurality of devices.

71. The chamber of claim 1 further comprising interface components that transmit data to a remote server.

72. The system of claim 71 wherein said remote server is selected from the group consisting of: a personal server, an institutional server, a proprietary server and a cloud server.

73. A chamber configured in size and shape to enclose a human subject, said chamber comprising:
a wall separating an internal gaseous environment for surrounding said subject from an external gaseous environment;
said wall featuring a slidable door or port to permit entry and exit of said human subject;
an internal array of field effect transistor (FET) nanosensor elements (NSEs) for sensing volatile compounds;
a mobile scanning ring inside said chamber that vertically scans said human subject by directing a gas towards said human subject and assaying volatile organic compounds (VOCs) mobilized by said directed gas to contact said internal array;
a computational system to collect, compile, store and analyze data resulting from said mobile scanner.

74. The chamber of claim 51 wherein said internal array of NSEs is static.

75. The chamber of claim 74 wherein said static internal array is located in a vertical structure bordering or defining limits of said walkway or standway.

76. The chamber of claim 74 wherein said static internal array is located above or below said walkway or standway.

77. The chamber of claim 75 wherein said vertical structure comprises an arch.

78-80. (canceled)

81. The chamber of claim 1 further comprising at least one accessory measurement device selected from the group consisting of: camera, thermometer, radiation detector, Geiger counter, x-ray, O₂ meter, pulse-ox meter, CO₂ meter, and scale to measure mass.
